(19) United States
(12) Patent Application Publication
(10) Pub. No.: US 2007/0036734 A1
(43) Pub. Date: Feb. 15, 2007

(54) Therapeutic agent for periodontal disease

(75) Inventors: Tomoyuki Tahara, Gunma (JP); Yoshimitsu Abiko, Tokyo (JP); Hiromasa Yoshie, Niigata (JP); Tetsuo Kobayashi, Niigata (JP); Toshio Umemoto, Kanagawa (JP); Nobushiro Hamada, Kanagawa (JP)

Correspondence Address: FOLEY AND LARDNER LLP;SUITE 500, 3000 K STREET NW, WASHINGTON, DC, 20007, US

(73) Assignees: Kirin Beer Kabushiki Kaisha; Nihon University

(21) Appl. No.: 10/549,289

(22) PCT Filed: Mar. 17, 2004
(86) PCT No.: PCT/JP04/03569

§ 371 Date: Aug. 14, 2006

(30) Foreign Application Priority Data
Mar. 17, 2003 (JP) 2003-072714

(51) Int. Cl. A61K 39/40; C07K 16/12; A61K 8/96; G01N 33/554; C12N 5/06

(52) U.S. Cl. 424/50; 435/7.32; 530/388.4; 424/164.1; 435/252.33; 435/326; 435/254.2

(57) ABSTRACT

The present invention provides a human monoclonal antibody that has both activity of inhibiting the aggregation of pathogenic bacteria involved in periodontal diseases and activity of promoting sterilization by leukocytes, that is preferably a monoclonal antibody against 40-kDa OMP, which inhibits the binding of hemin, and that causes no concerns such as side effects. The present invention further provides an agent that acts against periodontal diseases containing the monoclonal antibody. The present invention relates to an antibody binding to 40-kDa OMP or a functional fragment thereof having at least one of (1) activity of inhibiting the coaggregation of P. gingivalis, (2) activity of promoting human neutrophilic phagocytosis, and (3) activity of inhibiting the binding of hemin to 40-kDa OMP.

### TECHNICAL FIELD

The present invention relates to an agent that acts against periodontal diseases. Specifically, the present invention relates to a human monoclonal antibody against 40-kDa OMP, that has both activity of inhibiting the aggregation of pathogenic bacteria involved in periodontal diseases and activity of promoting sterilization by leukocytes, that preferably inhibits the binding of 40-kDa OMP to hemin, and that has a lower possibility of causing side effects than antibodies derived from non-human animals such as mice. The present invention also relates to an agent that acts against periodontal diseases containing such monoclonal antibody.

### BACKGROUND ART

Periodontal diseases affect about 80% or more people in Japan. Major causes of periodontal diseases are thought to be infection with oral bacteria, increases in pathogenic bacteria involved in periodontal diseases, invasion of tissues by bacteria, immune responses in hosts against infection, and the like. Periodontal diseases finally cause loss of teeth and thus are very important diseases that cause the quality of life to deteriorate. Recently, periodontal diseases have been shown to have causal relationships not only with tooth loss, but also with circulatory disorders, delivery of underweight babies and early delivery, diabetes, endocarditis, and pneumonia (Abiko Y., Crit. Rev. Oral. Biol. Med., 2000 Vol 11:140).

In therapeutic methods for periodontal diseases, elimination of the infection source is therapeutically important. However, extremely primitive methods including brushing and scaling, periodontal surgery, and the like are still major therapeutic methods for periodontal diseases. It is impossible to eradicate pathogenic bacteria involved in periodontal diseases with only such therapies involving mechanical elimination. Furthermore, it may be impossible to apply such therapies to patients with systemic disease, because bacillaemia or focal infection is also induced. Furthermore, administration of antibiotics (e.g., tetracycline and minocycline) or antibacterial agents has been considered to be effective periodontal therapy. However, current problems including multiple types of resistant bacteria, resistance-related genes, side effects, and the like have been indicated. Limitation of pharmacotherapy has also been suggested. Hence, in the present situation, no effective therapeutic methods have been established against periodontal diseases. The development of a new therapeutic method that is extremely safe for human bodies and enables eradication of pathogenic bacteria involved in periodontal diseases has been awaited.

Of oral bacteria, Porphyromonas gingivalis (P. gingivalis) has been isolated at high frequencies from the periodontal pockets of adult-type periodontal disease patients. P. gingivalis is thus the strongest suspect as a pathogenic bacteria involved in periodontal diseases. This bacterium is a gram-negative bacillus that forms black colonies on a blood plate. It is thought that the surface layer components of the bacterial body and extracellular products are responsible for disruption of periodontal tissues. Hence, to effectively prevent or treat periodontal diseases, it is important to suppress colonization of P. gingivalis so as to eliminate the bacterial bodies from within periodontal pockets.

As a method for destroying P. gingivalis, antibody therapy may be employed. Such antibody therapy involves preparing a specific antibody against a pathogenic factor of pathogenic bacteria involved in periodontal diseases and then administering the antibody to a periodontal pocket. By such antibody therapy, 1) suppression of pathogenic bacterial colonization in the periodontal pocket, 2) promotion of sterilization by leukocytes within periodontal pocket, and the like are expected. Regarding 1), it has been reported that although an antigen was unidentified, re-colonization of P. gingivalis could be successfully suppressed within the periodontal pocket for 9 months through local administration of an antibody against P. gingivalis (Booth, V. et al., Infect Immun., 1996 Vol 64: 422). As described above, periodontal diseases may be overcome to some extent through suppression of colonization of pathogenic bacteria involved in periodontal diseases using such specific antibody. Moreover, when it is impossible to apply invasive treatment to a case of severely infectious periodontal diseases, antibody therapy whereby activity of promoting sterilization (activity of promoting phagocytosis) by leukocytes within the periodontal pocket is actively expected may also be necessary.

Recently, 40-kDa outer membrane protein (OMP) has been reported as one of antigens expressed in P gingivalis (Abiko, Y. et. al., Arch. Oral. Biol. 1990 Vol 35: 689, Kawamoto, Y. et al., Int. J. Biochem., 1991 Vol 23: 1053). 40-kDa OMP is conserved among and expressed by many P. gingivalis strains (Hiratsuka, K. et. al., 1996, FEMS. Microbiol. Lett. 138:167-172). A monoclonal antibody against the 40-kDa OMP antigen inhibits the coaggregation of P. gingivalis and Actinomyces viscosus. The antigen is an important factor for colonization of P. gingivalis (Abiko, Y. et al., Infect. Immun., 1997 Vol 65: 3966; Hiratsuka, K. et al., Arch. Oral. Biol., 1992 Vol 37:717; Saito S. et al., Gen. Pharmacol., 1997 Vol 28:675). It has also been reported that an anti-40-kDa OMP polyclonal antibody has activity of promoting the phagocytic ability of a promyelocytic cell line HL60 (Saito, S. et al., J. Periodontol., 1999 Vol 70: 610). Hence, it is a great contribution to the development of a novel therapeutic method for periodontal diseases to provide a human monoclonal antibody that has activity of inhibiting the coaggregation of P. gingivalis and activity of promoting phagocytosis of P. gingivalis by leukocytes and that is excellent in terms of safety and long-lasting effects when it is applied to patients. However, no such antibodies have been reported. In general, it is not always clear whether all antibodies having activity of inhibiting the coaggregation of P. gingivalis have activity of promoting sterilization by leukocytes. Furthermore, it is known that incorporation of hemin is essential for the growth, proliferation, and exertion of the pathogenicity of P. gingivalis. In the 40-kDa OMP protein, a heme regulatory motif that is known as a hemin-binding site is present. It has been actually reported that 40-kDa OMP is a type of hemin-binding protein (Shibata, Y. et al., B.B.R.C., 2003 Vol 300:351). An antibody that inhibits the binding of 40-kDa OMP to hemin is likely to inhibit the growth and proliferation of P. gingivalis and strongly damages P. gingivalis.

### DISCLOSURE OF THE INVENTION

An object of the present invention is to provide an agent that acts against periodontal diseases, which is safe for human bodies while eliminating P. gingivalis, which is known as a pathogenic bacterium involved in periodontal diseases, from within the periodontal pocket. Particularly, an object of the present invention is to provide a human monoclonal antibody. Such human monoclonal antibody has both activity of inhibiting the aggregation of the above pathogenic bacteria involved in periodontal diseases and activity of promoting sterilization by leukocytes, is preferably a monoclonal antibody against 40-kDa OMP, which inhibits the binding of hemin, and further, has lower possibility of causing side effects than antibodies derived from non-human animals such as mice.

An object of the present invention is to achieve the above conventional objects. Specifically, we have conducted intensive studies to develop an agent that acts against periodontal diseases, which is safe for human bodies while eliminating P. gingivalis, which is known as pathogenic bacteria involved in periodontal diseases, from within the periodontal pocket. As a result, we have discovered that a human monoclonal antibody having both activity of inhibiting the aggregation of the pathogenic bacteria involved in periodontal diseases and activity of promoting sterilization by leukocytes, being preferably a monoclonal antibody against 40-kDa OMP, which inhibits the binding of hemin, and further, having lower possibility of causing side effects than antibodies derived from non-human animals such as mice has good effects as an agent that acts against periodontal diseases. Thus, we have completed the present invention.

The present invention is as follows:
[1] an antibody binding to 40-kDa OMP or a functional fragment thereof, which has activity of inhibiting the binding of hemin to 40-kDa OMP;
[2] an antibody binding to 40-kDa OMP or a functional fragment thereof, which has (1) activity of inhibiting the coaggregation of P. gingivalis and (2) activity of promoting human neutrophilic phagocytosis;
[3] an antibody binding to 40-kDa OMP or a functional fragment thereof, which has (1) activity of inhibiting the coaggregation of P. gingivalis and (2) activity of inhibiting the binding of hemin to 40-kDa OMP;
[4] an antibody binding to 40-kDa OMP or a functional fragment thereof, which has (1) activity of promoting human neutrophilic phagocytosis and (2) activity of inhibiting the binding of hemin to 40-kDa OMP;
[5] an antibody binding to 40-kDa OMP or a functional fragment thereof, which has (1) activity of inhibiting the coaggregation of P. gingivalis, (2) activity of promoting human neutrophilic phagocytosis, and (3) activity of inhibiting the binding of hemin to 40-kDa OMP;
[6] the antibody or the functional fragment thereof according to any one of [2], [3], and [5], wherein the coaggregation of P. gingivalis is coaggregation of P. gingivalis and Actinomyces viscosus;
[7] an antibody binding to 40-KDa OMP or a functional fragment thereof, which has activity of suppressing alveolar bone resorption;
[8] the antibody or the functional fragment thereof according to any one of [1] to [7], wherein the antibody is a human antibody;
[9] the antibody or the functional fragment thereof according to any one of [1] to [8], which is produced by a mouse-mouse hybridoma;
[10] the antibody or the functional fragment thereof according to any one of [1] to [9], wherein the antibody is a monoclonal antibody;
[11] the antibody or the functional fragment thereof according to any one of [1] to [10], which covalently or non-covalently binds to a therapeutic agent;
[12] the antibody or the functional fragment thereof according to [11], wherein the therapeutic agent is selected from antibiotics or antibacterial agents;
[13] the antibody or the functional fragment thereof according to [12], wherein the antibiotic or the antibacterial agent is tetracycline or minocycline;
[14] the antibody or the functional fragment thereof according to any one of [1] to [13], wherein the antibody class is IgG;
[15] the antibody or the functional fragment thereof according to [14], wherein IgG is IgG1;
[16] the antibody or the functional fragment thereof according to any one of [1] to [13], wherein the antibody class is IgA;
[17] The antibody or the functional fragment thereof according to any one of [1] to [16], wherein the amino acid sequence of a heavy chain constant region is altered;
[18] an antibody binding to 40-kDa OMP or a functional fragment thereof, which is produced by a hybridoma h13-17 (accession No. FERM BP-8325);
[19] an antibody binding to 40-kDa OMP or a functional fragment thereof, which comprises variable regions of an antibody that is produced by a hybridoma h13-17 (accession No. FERM BP-8325);
[20] the antibody or the functional fragment thereof according to [18] or [19], which covalently or non-covalently binds to a therapeutic agent;
[21] the antibody or the functional fragment thereof according to [20], wherein the therapeutic agent is selected from antibiotics or antibacterial agents;
[22] the antibody or the functional fragment thereof according to [21], wherein the antibiotic or the antibacterial agent is tetracycline or minocycline;
[23] the antibody or the functional fragment thereof according to any one of [18] to [22], wherein the antibody class is IgG;
[24] the antibody or the functional fragment thereof according to [23], wherein IgG is IgG1;
[25] the antibody or the functional fragment thereof according to any one of [18] to [22], wherein the antibody class is IgA;
[26] the antibody or the functional fragment thereof according to any one of [18] to [25], wherein the amino acid sequence of a heavy chain constant region is altered;
[27] a hybridoma h13-17 (accession No. FERM BP-8325);
[28] an antibody binding to 40-kDa OMP or a functional fragment thereof, which is produced by a hybridoma 5-89-2 (accession No. FERM BP-8323);
[29] an antibody binding to 40-kDa OMP or a functional fragment thereof, which comprises variable regions of an antibody that is produced by a hybridoma 5-89-2 (accession No. FERM BP-8323);
[30] the antibody or the functional fragment thereof according to [28] or [29], which covalently or non-covalently binds to a therapeutic agent;
[31] the antibody or the functional fragment thereof according to [30], wherein the therapeutic agent is selected from antibiotics or antibacterial agents;
[32] the antibody or the functional fragment thereof according to [31], wherein the antibiotic or the antibacterial agent is tetracycline or minocycline;
[33] the antibody or the functional fragment thereof according to any one of [28] to [32], wherein the antibody class is IgG;
[34] the antibody or the functional fragment thereof according to [33], wherein IgG is IgG1;
[35] the antibody or the functional fragment thereof according to any one of [28] to [32], wherein the antibody class is IgA;
[36] the antibody or the functional fragment thereof according to any one of [28] to [35], wherein the amino acid sequence of a heavy chain constant region is altered;
[37] a hybridoma 5-89-2 (accession No. FERM BP-8323);
[38] an antibody binding to 40-kDa OMP or a functional fragment thereof, which is produced by a hybridoma a44-1 (accession No. FERM BP-8324);
[39] an antibody binding to 40-kDa OMP or a functional fragment thereof, which comprises variable regions of an antibody that is produced by a hybridoma a44-1 (accession No. FERM BP-8324);
[40] the antibody or the functional fragment thereof according to [38] or [39], which covalently or non-covalently binds to a therapeutic agent;
[41] the antibody or the functional fragment thereof according to [40], wherein the therapeutic agent is antibiotics or antibacterial agents;
[42] the antibody or the functional fragment thereof according to [41], wherein the antibiotic or the antibacterial agent is tetracycline or minocycline;
[43] the antibody or the functional fragment thereof according to any one of [38] to [42], wherein the antibody class is IgG;
[44] the antibody or the functional fragment thereof according to [43], wherein IgG is IgG1;
[45] the antibody or the functional fragment thereof according to any one of [38] to [42], wherein the antibody class is IgA;
[46] the antibody or the functional fragment thereof according to any one of [38] to [45], wherein the amino acid sequence of a heavy chain constant region is altered;
[47] a hybridoma a44-1 (accession No. FERM BP-8324);
[48] a nucleic acid, which is possessed by a hybridoma selected from the group consisting of a hybridoma h13-17 (accession No. FERM BP-8325), a hybridoma 5-89-2 (accession No. FERM BP-8323), and a hybridoma a44-1 (accession No. FERM BP-8324) and encodes an antibody containing a variable region of an antibody produced by the above hybridoma or a functional fragment of the said antibody;
[49] a protein encoded by the nucleic acid according to [48], which is an antibody or a functional fragment thereof;
[50] an expression vector, which has the nucleic acid according to [48];
[51] a host, which has the expression vector according to [50];
[52] the host according to [51], which is selected from the group consisting of Escherichia coli, yeast cells, insect cells, mammalian cells, plant cells, and mammals;
[53] a method for producing an antibody binding to 40-kDa OMP, which comprises isolating a gene that encodes an antibody binding to 40-kDa OMP from a hybridoma selected from the group consisting of a hybridoma h13-17 (accession No. FERM BP-8325), a hybridoma 5-89-2 (accession No. FERM BP-8323), and a hybridoma a44-l (accession No. FERM BP-8324), constructing an expression vector comprising the gene, introducing the expression vector into a host to cause expression of the antibody, and collecting the antibody from the obtained host, the culture supernatant of the host, or secretion from the host;
[54] an agent for suppressing alveolar bone resorption, which contains an antibody binding to 40-KDa OMP or a functional fragment thereof as an active ingredient;
[55] an agent for preventing, diagnosing, or treating periodontal diseases, which contains an antibody binding to 40-kDa OMP or a functional fragment thereof as an active ingredient;
[56] use of an antibody binding to 40-KDa OMP or a functional fragment thereof for production of an agent for suppressing alveolar bone resorption;
[57] a method for suppressing alveolar bone resorption, which comprises preparing an antibody binding to 40-KDa OMP or a functional fragment thereof and administering the antibody or the fragment to an animal;
[58] use of an antibody binding to 40-KDa OMP or a functional fragment thereof for production of an agent for preventing, diagnosing, or treating periodontal diseases;
[59] a method for diagnosing, preventing, or treating periodontal diseases, which comprises preparing an antibody binding to 40-KDa OMP or a functional fragment thereof and administering the antibody or the fragment to an animal;
[60] an agent for preventing, diagnosing, or treating periodontal diseases, which contains the antibody or the functional fragment thereof according to any one of [1] to [26], [28] to [36], [38] to [46], and [49] as an active ingredient;
[61] an agent for suppressing alveolar bone resorption, which contains the antibody or the functional fragment thereof according to any one of [1] to [26], [28] to [36], [38] to [46], and [49] as an active ingredient;
[62] use of the antibody or the functional fragment thereof according to any one of [1] to [26], [28] to [36], [38] to [46], and [49] for production of an agent for preventing, diagnosing, or treating periodontal diseases;
[63] use of the antibody or the functional fragment thereof according to any one of [1] to [26], [28] to [36], [38] to [46], and [49] for production of an agent for suppressing alveolar bone resorption;
[64] a method for diagnosing, preventing, or treating periodontal diseases, which comprises preparing the antibody or the functional fragment thereof according to any one of [1] to [26], [28] to [36], [38] to [46], and [49] and administering the antibody or the fragment to an animal; and
[65] a method for suppressing alveolar bone resorption, which comprises preparing the antibody or the functional fragment thereof according to any one of [1] to [26], [28] to [36], [38] to [46], and [49] and administering the antibody or the fragment to an animal.

This description includes part or all of the contents as disclosed in the description and/or drawings of Japanese Patent Application No. 2003-072714, which is a priority document of the present application.

### BRIEF DESCRIPTION OF THE DRAWINGS

FIG. 1 shows the reactivity of each anti-40kDa OMP antibody against r40-kDa OMP and P. gingivalis.

FIG. 2 shows a comparison of the binding activity of each anti-40-kDa OMP antibody and the serum of a periodontal disease patient to P. gingivalis.

FIG. 3 shows the results of analyzing reactions between each anti-40-kDa OMP antibody and P. gingivalis.

FIG. 4 shows the effect of each anti-40-kDa OMP antibody on the interaction between 40-kDa OMP and hemin.

FIG. 5 shows an h13-17 antibody's activity of inhibiting the binding of 40-kDa OMP to hemin.

FIG. 6 shows the reactivity of each anti-40-kDa OMP antibody to various P. gingivalis strains.

FIG. 7 shows each anti-40-kDa OMP antibody's activity of suppressing rat alveolar bone resorption.

### BEST MODE OF CARRYING OUT THE INVENTION

The present invention will be described in detail as follows.

40-kDa OMP can be produced by appropriate employment of a method known in the technical field, such as a chemical synthesis method or a cell culture method, in addition to a gene recombination technique based on a known nucleotide sequence (DNA Data Bank of Japan: accession No. AB059658) or a known amino acid sequence. The nucleotide sequence of 40-kDa OMP is shown in SEQ ID NO: 1 and the amino acid sequence of 40-kDa OMP is shown in SEQ ID NO: 2. Furthermore, a partial sequence of 40-kDa OMP can also be produced according to a method known in the technical field and described later, such as a gene recombination technique or a chemical synthesis method. 40-kDa OMP can be produced by appropriate cleavage using proteinase or the like.

The antibody or the functional fragment thereof of the present invention encompasses various anti-40-kDa OMP monoclonal antibodies or functional fragments thereof having the following reactivities. Specifically, (1) an antibody binding to 40-kDa OMP or a functional fragment thereof having activity of inhibiting the coaggregation of P. gingivalis and activity of promoting human neutrophilic phagocytosis, (2) an antibody binding to 40-kDa OMP or a functional fragment thereof having activity of inhibiting the coaggregation of P. gingivalis, activity of promoting human neutrophilic phagocytosis, and activity of inhibiting the binding of hemin to 40-kDa OMP, (3) an antibody binding to 40-kDa OMP or a functional fragment thereof having activity of promoting human neutrophilic phagocytosis and activity of inhibiting the binding of hemin to 40-kDa OMP, (4) an antibody binding to 40-kDa OMP or a functional fragment thereof having activity of inhibiting the coaggregation of P. gingivalis and activity of inhibiting the binding of hemin to 40-kDa OMP, or (5) an antibody binding to 40-kDa OMP or a functional fragment thereof having activity of inhibiting the binding of hemin to 40-kDa OMP are encompassed.

“Coaggregation of P. gingivalis” indicates the aggregation of P. gingivalis with other microorganisms such as Actinomyces viscosus and Streptococcus gordonii. Such aggregation results in colonization of pathogenic bacteria in the form of plaques in the periodontal pocket. Accordingly, “activity of inhibiting the aggregation of P. gingivalis” of the antibody of the present invention indicates activity by which aggregation of P. gingivalis with other bacteria can be inhibited. Whether or not an antibody has such activity of inhibiting aggregation can be determined by a method described in Example 9 in this description. When activity of inhibiting aggregation is measured by the method described in Example 9, the score of the antibody of the present invention is preferably 2 or less. Furthermore, “activity of promoting sterilization by leukocytes” of the antibody of the present invention indicates activity of promoting the phagocytosis of P. gingivalis by leukocytes such as neutrophils. Such activity can be determined by a method described in Example 10 in this description. When such activity of promoting phagocytosis is measured by the method described in Example 10, the phagocytosis rate in the case of the antibody of the present invention is significantly higher than that in the case of a control antibody. Furthermore, the antibody of the present invention encompasses an antibody having activity of inhibiting the binding of P. gingivalis 40-kDa OMP to hemin. Whether or not such antibody inhibits the binding of P. gingivalis 40-kDa OMP to hemin can be determined by the method described in Example 14 in this description. When such activity of inhibiting the binding to hemin is measured by the method described in Example 14, the activity of the antibody of the present invention is significantly higher than that of a control antibody. P. gingivalis is used for verification of the activity of the antibody of the present invention. Any P. gingivalis may be used, as long as it expresses 40-kDa OMP. Examples of such P. gingivalis include P. gingivalis 381 used in the Examples of the present invention and P. gingivalis W50 (ATCC No. 53978).

Examples of such antibody or a functional fragment thereof include an anti-40-kDa OMP monoclonal antibody described later and a monoclonal antibody comprising a heavy chain and/or a light chain having an amino acid sequence derived from the amino acid sequence of a heavy chain and/or a light chain composing the antibody by deletion, substitution, or addition of 1 or several amino acids and having the reactivity according to any one of (1) to (5) above. “Alteration” (deletion, substitution, insertion, and addition) of amino acids as described above can be introduced by partially altering the nucleotide sequence encoding a relevant amino acid sequence. For example, such partial alteration in a nucleotide sequence can be introduced by a standard method using known site-specific mutagenesis (Proc Natl Acad Sci U.S.A., 1984 81: 5662; Sambrook et al., Molecular Cloning A Laboratory Manual (1989) Second edition, Cold Spring Harbor Laboratory Press). For example, an antibody wherein the amino acid sequence of a heavy chain constant region has been altered in the present invention may have stronger activity of promoting phagocytosis of P. gingivalis by leukocytes than that of the antibody before alteration because of improved affinity for an Fc receptor.

“Antibody” of the present invention also encompasses an antibody having any immunoglobulin class and subclass and is preferably an antibody having human immunoglobulin class and subclass. Examples of preferable classes (and subclasses) are immunoglobulin G (IgG) and IgA, and particularly IgG1 and IgA.

Another preferable example of the antibody or the fragment thereof of the present invention is a sequence composing a monoclonal antibody or a fragment thereof, which recognizes an epitope in the amino acid sequence of 40-kDa OMP and has the reactivity according to any one of (1) to (5) above.

A fragment of an antibody in the present invention means a portion of the antibody as defined above. Specific examples of such fragment include F(ab′)2, Fab′, Fab, Fv, disulphide-linked FV, Single-Chain FV (scFV), and polymers thereof (D. J. King., Applications and Engineering of Monoclonal Antibodies., 1998 T. J. International Ltd). Such antibody fragment can be obtained by a conventional method, including digestion of an antibody molecule with protease such as papain or pepsin or by a known genetic engineering technique. “Functional fragment” means a fragment of an antibody that specifically binds to an antigen to which a complete antibody specifically binds.

The antibody of the present invention can be produced by the following method, for example. Specifically, for example, a non-human mammal (e.g., a mouse, a rabbit, a goat, or a horse) is immunized with 40-kDa OMP as defined above or a portion thereof, a product obtained by binding with an appropriate substance (e.g., bovine serum albumin) for enhancing the antigenicity of an antigen, or a cell expressing 40-kDa OMP on the cell surfaces in large quantities, together with an immuno-augmenting agent (e.g., Freund's adjuvant), if necessary. Alternatively, an expression vector into which 40-kDa OMP has been incorporated is administered to a non-human mammal, so that immunization can be carried out. A monoclonal antibody is produced by preparing a hybridoma of an antibody-producing cell obtained from an immunized animal and a cell of a myeloma cell line (myeloma cell) incapable of producing any autoantibody, cloning the hybridoma, and then selecting clones that produce monoclonal antibodies showing specific affinity for an antigen used for immunization. More preferably, through the use of a non-human animal that retains an unrearranged human antibody gene and produces a human antibody specific to the relevant immunogen as a result of immunization, the antibody of the present invention can be obtained as a human antibody. An example of such non-human animal is a mouse. A method for producing a mouse capable of producing a human antibody is described in International Patent Publication W002/43478. Here, “human antibody” means an antibody that is an expression product of a human-derived antibody gene or means a functional fragment thereof. Examples of the monoclonal antibody of the present invention include monoclonal antibodies produced by hybridoma clones h13-17 (accession No. FERM BP-8325), 5-89-2 (FERM BP-8323), and a44-1 (FERM BP-8324) that were internationally deposited at the International Patent Organism Depositary of the National Institute of Advanced Industrial Science and Technology (Tsukuba Central 6, 1-1-1 Higashi, Tsukuba, Ibaraki, Japan) as of Mar. 11, 2003, under the Budapest Treaty, or functional fragments thereof. Moreover, antibodies obtained by alteration of the classes or subclasses of these monoclonal antibodies are also included. Furthermore, antibodies obtained by altering the amino acid sequences of the heavy chain constant regions of antibodies produced by these hybridomas or functional fragments thereof are also included.

The present invention also encompasses a nucleic acid that is possessed by the above hybridoma and encodes an antibody containing a variable region of an antibody produced by the hybridoma and a nucleic acid encoding a functional fragment of such antibody. These nucleic acids can be obtained by a general genetic engineering technique from hybridomas. The nucleotide sequences thereof can also be determined by a known method for determining nucleotide sequences. Furthermore, the present invention also includes proteins encoded by nucleic acids obtained as described above. Such proteins have reactivity according to any one of (1) to (5) above. Moreover, the present invention also encompasses expression vectors containing the above nucleic acids and host cells containing such expression vectors. Such vectors and host cells are not limited. Examples of such hosts include not only Escherichia coli, yeast cells, insect cells, mammalian cells, and plant cells, but also include insect individuals and mammalian individuals. Examples of insect individuals include silkworms and examples of mammalian individuals include mice, rats, cattle, horses, sheep, and pigs, but are not limited thereto. As expression vectors, known vectors and commercial vectors corresponding to each host can be used. Furthermore, insect individuals or mammalian individuals can be transfected by known methods using expression vectors. The present invention further encompasses a method for producing the antibody or the functional fragment thereof of the present invention, which comprises causing expression of the nucleic acid of the present invention in host cells or host individuals that contain an expression vector containing the nucleic acid and collecting the antibody or the functional fragment thereof (expression product) from the culture solution of the host cells, a host body fluid, or a host secretion such as milk.

Specifically, the antibody or the functional fragment thereof of the present invention can be produced as follows. A hybridoma secreting a monoclonal antibody can be prepared by the method of Kohler and Milstein et al. (Nature., 1975 Vol. 256: 495-497), or according to such method. Specifically, antibody-producing cells are contained in the spleen, lymph node, bone marrow, tonsil, or the like, or preferably the lymph node or the spleen obtained from an animal immunized as described above. Such antibody-producing cells are fused with myeloma cells that are derived from a mammal and preferably from a mouse, rat, guinea pig, hamster, rabbit, human, or the like and are incapable of producing any autoantibody. Thus, the antibody or the functional fragment thereof of the present invention can be prepared. For example, cell fusion can be carried out by mixing antibody-producing cells with niyeloma cells in a high-concentration polymer solution of polyethylene glycol or the like (e.g., with a molecular weight ranging from 1500 to 6000) generally at a temperature ranging from approximately 30° C. to 40° C. for approximately 1 to 10 minutes. Screening for hybridoma clones producing monoclonal antibodies can be carried out as follows. For example, hybridomas are cultured on a microtiter plate. Reactivity of a culture supernatant from well in which the proliferation of the hybridoma has been obserbed to an antigen for immunization is measured using an immunological method such as the enzyme-linked immunoassay (e.g., ELISA), radioimmunoassay, or fluorescent antibody technique.

A monoclonal antibody can be produced from a hybridoma by culturing in vitro hybridomas and then isolating monoclonal antibodies from a culture supernatant. Furthermore, hybridomas can be cultured in vivo in the ascite or the like of a mouse, rat, guinea pig, hamster, rabbit, or the like and then the monoclonal antibodies can also be isolated from the ascite.

Furthermore, a recombinant antibody can be prepared by a gene recombination technique, specifically by cloning a gene encoding a monoclonal antibody from an antibody-producing cell such as a hybridoma, incorporating the resultant into an appropriate vector, and then introducing the vector into a host (e.g., a cell of a mammalian cell line such as a Chinese hamster ovary (CHO) cell, Escherichia coli, yeast cell, insect cell, or plant cells) (P. J. Delves., ANTIBODY PRODUCTION ESSENTIAL TECHNIQUES., 1997 WILEY, P. Shepherd and C. Dean., Monoclonal Antibodies., 2000 OXFORD UNIVERSITY PRESS, J. W. Goding., Monoclonal Antibodies: principles and practice., 1993 ACADEMIC PRESS). Moreover, transgenic cattle, goats, sheep, or pigs wherein a target antibody gene is incorporated in an endogenous gene can be produced using a transgenic animal production technique. Monoclonal antibodies derived from the antibody gene can also be obtained in large quantities from the milk of the transgenic animals. Hybridomas can be cultured in vitro as follows. In accordance with various conditions including the properties of cell species to be cultured, the purposes of tests and studies, culture methods, and the like, known nutrition media or every nutrition media that are induced and prepared from known basic media are used for culture. Such media enable proliferation, maintenance, and storage of hybridomas and are used for causing the hybridomas to produce monoclonal antibodies in culture supernatants.

The thus produced monoclonal antibodies can be purified by appropriate combination of methods known in the art, such as chromatography using a protein A column or protein G column, ion exchange chromatography, hydrophobic chromatography, an ammonium sulfate salting out method, gel filtration, and affinity chromatography.

The monoclonal antibody or the fragment thereof of the present invention produced by the above method can be used to form a complex. Such complex can be used for treatment such as missile therapy by conjugating the antibody or the fragment thereof to a drug for treatment. Examples of such drug for treatment to be conjugated to an antibody include, but are not limited to, antibiotics such as tetracycline and minocycline and antibacterial agents. An antibody may be conjugated to a drug for treatment via either covalent or non-covalent bonding (e.g., ionic bonding). For example, a reactive group (e.g., an amino group, a carboxyl group, and a hydroxyl group) in an antibody molecule or a coordination group is used. Specifically, the complex of the present invention can be obtained by causing an antibody to come into contact with a drug for treatment containing a functional group (in the case of bacterial toxin and a chemical therapeutic agent) capable of reacting with the reactive group or containing an ionic group (in the case of a radioactive nuclide) capable of forming a complex with the coordination group. Alternatively, a biotin-avidin system can also be used for the formation of such complex. Furthermore, when a drug for treatment is a protein or a peptide, the complex of the present invention can also be produced as a fusion protein of an antibody with the above protein or peptide by a genetic engineering technique.

Moreover, the anti-40-kDa OMP antibody of the present invention or a pharmaceutical composition for preventing, diagnosing, or treating periodontal diseases, which contains the anti-40-kDa OMP antibody conjugated to a drug for treatment as described above, is also included within the scope of the present invention. Through administration of the anti-40-kDa OMP antibody of the present invention, P. gingivalis can be eliminated from the oral cavity. Furthermore, disruption of periodontal tissues due to P. gingivalis can be prevented and periodontal diseases can be prevented and treated. Such composition should contain the therapeutically effective dose of a drug for treatment and is formulated into various forms for oral and parenteral administration. Here, “therapeutically effective dose” indicates an amount of the composition that can have therapeutic effects on given symptoms or can improve administration plans. The composition of the present invention can contain, in addition to an antibody, 1 type or a plurality of types of physiologically acceptable pharmaceutical additives such as a diluent, a preservative, a solubilizing agent, an emulsifier, an adjuvant, an antioxidant, an isotonizing agent, an excipient, and a carrier. Furthermore, the composition of the present invention may also be prepared as part of a mixture with another antibody or another drug such as an antibiotic. Examples of an appropriate carrier include, but are not limited to, a physiological saline solution, phosphate buffered saline, a phosphate buffered saline glucose solution, and a buffered saline solution. Furthermore, the composition may also contain a stabilizer, such as amino acids, sugars, a surfactant, or an agent for preventing adsorption to the surface, which are known in the art. Examples of such form of a pharmaceutical preparation include a paste, a liquid, a freeze-dried pharmaceutical preparation (in this case, the preparation can be reshaped by the addition of a buffered aqueous solution as described above and then used), a sustained release pharmaceutical preparation, an enteric coated pharmaceutical preparation, an injection, and drops. The form of pharmaceutical preparation can be selected from these examples according to treatment purposes and treatment plans.

Possible routes of administration are oral administration and non-enteral administration including intravenous, intramuscular, subcutaneous, and intraperitoneal injections or drug delivery. An optimum route is selected based on tests using animals. Alternatively, another possible method involves causing the composition of the present invention to come into direct contact with an affected region of a patient. In view of direct application of the composition to a region affected by periodontal diseases, it is preferable to administer the composition into the oral cavity or the periodontal pocket. The dose is appropriately determined by implementation of tests using animals and clinical tests. In general, conditions or severity, age, body weight, sex, and the like of a patient should be considered.

Furthermore, the antibody or the functional fragment of the present invention may be mixed into toothpaste or an intraoral wash and then applied to a region affected by periodontal diseases. The antibody or the fragment thereof can also be applied in the form of a functional food by mixing the antibody or the fragment into food, drink, or the like.

Furthermore, the antibody or the functional fragment thereof of the present invention can also be used as a diagnostic agent for periodontal diseases. For example, whether or not P. gingivalis is present in the periodontal pocket can be detected using the antibody or the functional fragment thereof of the present invention. Such detection can be carried out by collecting plaque in the periodontal pocket and then detecting the presence of P. gingivalis in the plaque by a known immunoassay such as EIA, RIA, or an immunoagglutination method.

Hereafter, the present invention is described in detail by referring to examples, but is not limited to embodiments described in the examples.

### EXAMPLE 1

### Preparation of Recombinant 40-kDa OMP (r40-kDa OMP)

Recombinant 40-kDa OMP (r40-kDa OMP) was prepared as follows. Escherichia coli (K-12) having a recombinant plasmid pMD125 prepared by incorporation of full-length r40-kDa OMP DNA (DNA Data Bank of Japan: accession No. AB059658) into a vector was cultured in an LB medium (1% tryptone (produced by Becton, Dickinson and Company), 0.5% yeast extract (produced by Becton, Dickinson and Company), and 0.5% NaCl) containing 10 μ/mL tetracycline. Bacterial bodies were collected using a centrifuge and then disrupted by ultrasonication. A supernatant wherein the bacterial bodies had been disrupted was obtained using a centrifuge and then r40-kDa OMP was purified according to the method of Kawamoto et al., (Int. J. Biochem.1991 Vol 23: 1053). The thus prepared r40-kDa OMP was subjected to substitution with PBS(−) using a dialysis membrane (molecular weight of 10,000 or less as a cut-off, produced by Spectrum Laboratories Inc.). Thus, a purified protein of a single band corresponding to a molecular weight of 40,000 was obtained by SDS/PAGE electrophoresis.

### EXAMPLE 2

### Production of Human-antibody-producing Mice

Mice used for immunization have genetic background such that they are homozygous for both disruption in the endogenous Ig heavy chain and disruption in the κ light chain. Furthermore, the mice simultaneously retained a chromosome 14 fragment (SC20) containing a human Ig heavy chain locus and a human Igκ chain transgene (KCo5). These mice were produced by crossing mice of a line A having a human Ig heavy chain locus with mice of a line B having a human Igκ chain transgene. The mice of the line A are homozygous for both disruption in the endogenous Ig heavy chain and disruption in the κ light chain. Furthermore, the mice of the line A retain a chromosome 14 fragment (SC20) that can be transferred to progeny and is described in the report of Tomizuka et al. (Tomizuka. et al., Proc. Nati. Acad. Sci. U.S.A., 2000 Vol 97: 722), for example. Furthermore, the mice of the line B are homozygous for both disruption in the endogenous Ig heavy chain and disruption in the κ light chain. The mice of the line B retain a human IgK chain transgene (KCo5) and are described in the report of Fishwild et al., (Nat. Biotechnol., 1996 Vol 14: 845), for example. Mouse individuals (Ishida & Lonberg, IBC's 11th Antibody Engineering, Abstract 2000) for which the human Ig heavy chain and the K light chain are simultaneously detected in serum were obtained by crossing male mice of the line A with female mice of the line B or crossing female mice of the line A with male mice of the line B. Such mice were used in the following immunological experiments. In addition, the above human-antibody-producing mice can be acquired from Kirin Brewery Co., Ltd. based on conclusion of a contract.

### EXAMPLE 3

### Preparation of a Human Monoclonal Antibody Against 40-kDa OMP

Monoclonal antibodies in this example were prepared according to general methods described in “Introduction to Monoclonal Antibody Experimental Protocols (Monoclonal Ko-tai Jikken So-sa Nyu-mon) (written by Tamie Ando, issued by KODANSHA, 1991) and the like. r40-kDa OMP prepared in Example 1 was used as an immunogen. As animals to be immunized, the human-antibody-producing mice (produced in Example 2) producing human immunoglobulin were used.

r40-kDa OMP prepared in Example 1 was mixed with an RIBI adjuvant (produced by Corixa Corporation). The human-antibody-producing mice were subjected to initial immunization by intraperitoneal administration of 20 μg of r40-kDa OMP. Booster immunization was carried out by 4 times intraperitoneal administration of the mixed solution of r40-kDa OMP and the RIBI adjuvant every 1 to 2 weeks after initial immunization. Furthermore, 3 days before obtaining the spleen cells as described below, booster immunization was carried out by tail intravenous injection of r40-kDa OMP.

Spleens were surgically obtained from the immunized mice. The thus collected spleen cells were mixed with mouse myeloma SP2/0 (ATCC No.: CRL1581) at a ratio of 5:1. Thus, cell fusion was carried out using polyethylene glycol 1500 (produced by Boehringer Mannheim) as a fusion agent, thereby preparing many hybridomas. Hybridomas were selected by culturing them in a HAT-containing DMEM medium (produced by Gibco BRL) containing 10% fetal calf serum (FCS), hypoxanthine (H), aminopterin (A), and thymidine (T). Furthermore, single clones were prepared by a limiting dilution method using an HT-containing DMEM medium. Culture was carried out in a 96-well microtiter plate (produced by Becton, Dickinson and Company). Selection (screening) of hybridoma clones producing anti-r40-kDa OMP human monoclonal antibodies and characterization of a human monoclonal antibody produced by each hybridoma were carried out by measurement such as enzyme linked immunosorbent assay (ELISA) or fluorescence activated cell sorting (FACS) described later.

Human-monoclonal-antibody-producing hybridomas were screened for by ELISA, specifically by enzyme linked immunosorbent assay (ELISA) and fluorescence activated cell sorting (FACS) described below. Hybridomas producing human monoclonal antibodies that have a human immunoglobulin γ chain (hIgγ), a human immunoglobulin light chain κ, and specific reactivity to r40-kDa OMP were obtained. In addition, in any of the following Examples, including this Example, and in tables or figures given to show test results in the Examples, hybridoma clones producing each human anti-40-kDa OMP monoclonal antibody of the present invention were denoted using symbols. The following hybridoma clones represent single clones: h13-17, 5-89-2, a44-1, and 1-85-16. Of these clones, 3 hybridoma clones, h13-17, 5-89-2, and a44-1, were internationally deposited at the International Patent Organism Depositary of the National Institute of Advanced Industrial Science and Technology (Tsukuba Central 6, 1-1-1 Higashi, Tsukuba, Ibaraki, Japan) under the Budapest Treaty. The accession nos. of the hybridoma clones h13-17, 5-89-2, and a44-1 are FERM BP-8325, FERM BP-8323, and BP-8324, respectively (as of Mar. 11, 2003).

### EXAMPLE 4

### Detection of a Monoclonal Antibody having a Human Immunoglobulin γ Chain

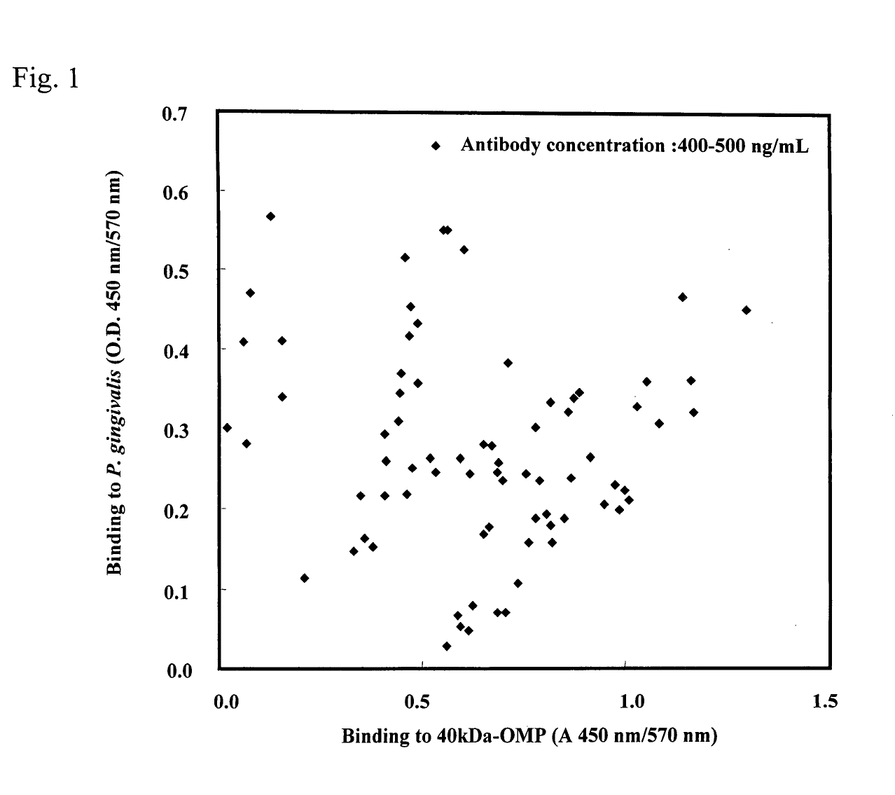

50 μl of r40-kDa OMP (1 μg/ml 50 mM Na₂HCO₃) prepared in Example 1 was added to each well of a 96-well microplate for ELISA (Maxisorp, produced by Nunc), followed by 30 minutes of incubation at room temperature. r40-kDa OMP was thus adsorbed onto the microplate. Subsequently, the supernatants were discarded. A blocking reagent (SuperBlock™ Blocking Buffer produced by Pierce Biotechnology Inc.,) was added to each well, followed by 10 minutes of incubation at room temperature. Thus, sites to which no r40-kDa OMP had bound were blocked. In this manner, a microplate was prepared so that each well was coated with r40-kDa OMP. Furthermore, P. gingivalis 381 strain was anaerobically cultured in Tripticase soy broth (produced by BBL) supplemented with 5 μg/mL hemin (produced by Sigma), 0.5 μg/mL vitamin K, and 0.5% yeast extract (produced by Difco Laboratories) at 37° C. P. gingivalis was grown to the mid-log phase. Subsequently, the bacterial cells were collected by centrifugation (10,000×g, 10 minutes, and 4° C.), followed by heat treatment at 60° C. for 30 minutes. Next, the cells were re-suspended in PBS and then subjected to sonication on ice for 15 minutes using an ultrasonic homogenizer (Branson Sonifier 250). The ultrasonicated resultant was centrifuged (100,000×g, 30 minutes, and 4° C.) and then the supernatant was filtered (0.22 μm). The concentration of the ultrasonicated resultant was found by measurement of absorbance at 280 nm and then calculation where 1 mg/ml was determined to be an optimal density (O.D.) of 1.0. Ultrasonicated P. gingivalis (50 μg/ml 50 mM Na₂HCO₃ and 50 μl/well) was added to each well of a 96-well microplate for ELISA (Maxisorp , produced byNunc), followed by 30 minutes of incubation at room temperature. Thus, the ultrasonicated P. gingivalis was adsorbed to the microplate. Subsequently, supernatants were discarded. A blocking reagent (SuperBlock™ Blocking Buffer produced by Pierce Biotechnology Inc.,) was added to each well, followed by 10 minutes of incubation at room temperature. Each well was washed twice with 0.1% Tween20-containing phosphate buffer (PBS-T). 50 μl of the culture supernatant of each hybridoma was added to each well of the microplate coated with r40-kDa OMP or ultrasonicated P. gingivalis, followed by 30 minutes of reaction at room temperature. Each well was then washed twice with PBS-T. A goat anti-human IgG F(ab′)² antibody (produced by Biosource International) labeled with peroxidase was diluted 2000-fold by using PBS-T containing 10% Block Ace (produced by Dainippon Pharmaceutical Co., Ltd.). 50 μl of the solution was added to each well, followed by 30 minutes of incubation at room temperature. The microplate was washed three times with PBS-T. 100 μl of a coloring substrate solution (TMB produced by DAKO Corporation) was added to each well, followed by 20 minutes of incubation at room temperature. 50 μl of 2 M sulfuric acid was added to each well so as to stop the reaction. Absorbance at a wavelength of 450 nm (reference wavelength: 570 nm) was measured using a microplate reader (MTP-300 produced by Corona Electric Co., Ltd). As a result, 300 or more anti-r40-kDa OMP antibody clones could be obtained. Some of the clones are shown in FIG. 1.

### EXAMPLE 5

### Detection of a Monoclonal Antibody having Human Immunoglobulin Light Chain κ (IgLκ)

50 μl of r40-kDa OMP (1 μg/ml 50 mM Na₂HCO₃) prepared in Example 1 was added to each well of a 96-well microplate for ELISA (Maxisorp, produced by Nunc), followed by 30 minutes of incubation at room temperature. r40-kDa OMP was thus adsorbed onto the microplate. Subsequently, the supernatants were discarded. A blocking reagent (SuperBlock™ Blocking Buffer produced by Pierce Biotechnology Inc.) was added to each well, followed by 10 minutes of incubation at room temperature. Each well was washed twice with PBS-T. 50 μl of the culture supernatant of each hybridoma was added to each well of the microplate coated with r40-kDa OMP, followed by 30 minutes of reaction. Each well was then washed twice with PBS-T. 50 μl of a goat anti-human Igκ antibody (diluted 2,000-fold and produced by Biosource International) labeled with peroxidase was added to each well, followed by 30 minutes of incubation at room temperature. After washing three times with PBS-T, 100 μl of a substrate buffer (TMB produced by DAKO Corporation) was added to each well, followed by 20 minutes of incubation at room temperature. 50 μl of 2 M sulfuric acid was added to each well so as to stop the reaction. Absorbance at a wavelength of 450 nm (reference wavelength: 570 nm) was measured using a microplate reader (MTP-300 produced by Corona Electric Co., Ltd).

### EXAMPLE 6

### Identification of the Subclass of each Monoclonal Antibody

50 μl of r40-kDa OMP (1 μg/ml 50 mM Na₂HCO₃) prepared in Example 1 was added to each well of a 96-well microplate for ELISA (Maxisorp, produced by Nunc), followed by 30 minutes of incubation at room temperature. r40-kDa OMP was thus adsorbed onto the microplate. Subsequently, the supernatants were discarded. A blocking reagent (SuperBlock™ Blocking Buffer produced by Pierce Biotechnology Inc.) was added to each well, followed by 10 minutes of incubation at room temperature. Each well was washed twice with PBS-T. 50 μl of the culture supernatant of each hybridoma was added to each well of the microplate coated with r40-kDa OMP, followed by 30 minutes of reaction. Each well was then washed twice with PBS-T. Subsequently, 50 μl of a sheep anti-human IgG1 antibody, a sheep anti-human IgG2 antibody, a sheep anti-human IgG3 antibody, or a sheep anti-human IgG4 antibody (diluted 2,000-fold and produced by The Binding Site Limited) labeled with peroxidase was added to each well, followed by 30 minutes of incubation at room temperature. After washing three times with PBS-T, 100 μl of a substrate buffer (TMB produced by DAKO Corporation) was added to each well, followed by 20 minutes of incubation at room temperature. 50 μl of 2 M sulfuric acid was then added to each well so as to stop the reaction. Absorbance at a wavelength of 450 nm (reference wavelength: 570 nm) was measured using a microplate reader (MTP-300 produced by Corona Electric Co., Ltd).

### EXAMPLE 7

### Preparation of each Antibody

Culture supernatants containing the anti-r40-kDa OMP antibodies were prepared by the following method. Anti-r40-kDa OMP antibody-producing hybridomas were adapted to an eRDF medium (produced by KYOKUTO PHARMACEUTICAL INDUSTRIAL CO., LTD.) containing cattle insulin (5 μg/ml, produced by Gibco BRL), human transferrin (5 μg/ml, produced by Gibco BRL), ethanolamine (0.01 mM, produced by Sigma), and sodium selenite (2.5×10⁻⁵ mM, produced by Sigma). The hybridomas were cultured in a spinner flask. When the viable cell ratio of the hybridomas reached 90%, a culture supernatant was collected. The collected supernatant was filtered using a 10 μm filter and a 0.2 μm filter (produced by Gelman Science, Inc.), thereby removing miscellaneous substances including hybridomas and the like.

The anti-r40-kDa OMP antibodies were purified from the above culture supernatant by the following method. The culture supernatant containing the anti-r40-kDa OMP antibodies were subjected to affinity purification using the Hyper D Protein A column (produced by NGK INSULATORS, LTD.), PBS(−) as an adsorption buffer according to the attached instructions, and 0.1 M sodium citrate buffer (pH 3.5) as an elution buffer. The thus eluted fraction was adjusted to around pH 7.2 by the addition of 1 M Tris-HCl (pH 8.0). The thus prepared antibody solution was subjected to substitution with PBS(−) using a dialysis membrane (molecular weight of 10000 as a cut-off, produced by Spectrum Laboratories, Inc.,) and then filter-sterilized using a MILLEX-GV membrane filter (produced by Millipore Corporation) with a pore size of 0.22 μm. Thus, purified anti-r40-kDa OMP antibodies were obtained. The concentration of the purified antibodies was found by measurement of absorbance at 280 nm and calculation where 1 mg/ml was determined to be 1.45 O.D.

### EXAMPLE 8

### Preparation of Isotype Control Antibodies

In a manner similar to the above method, human-antibody-producing mice were immunized with DNP-KLH and then the spleen cells of the mice were fused with mouse myeloma SP2/0 cells. Thus, many hybridomas were prepared. Specifically, anti-human IgG1, IgG2, and IgG4 antibodies were prepared.

### EXAMPLE 9

### Inhibition of Co-aggregation of P. gingivalis by anti-r40-kDa OMP Antibody

A test of inhibiting the coaggregation of P. gingivalis was implemented by a modified version of the method of Ellen R. P. et al. (Infect. Immun. 1989; 57: 1618-1620). P. gingivalis 381 ((1) Postal code: 271-8587, 2-870-1, Nishi, Sakae-machi, Matsudo-shi, Chiba, Japan, Prof. Nobumitsu Abiko, Department of Biochemistry and Molecular Dentistry, NIHON UNIVERSITY School of Dentistry at Matsudo or (2) Postal code: 951-8514, 5274, 2-ban-cho, Gakko-machi-do-ri, Niigata-shi, acquired from Prof. Hiromasa Yoshie, Department of Oral Biological Science, Division of Periodontology, Niigata University Graduate School of Medical and Dental Sciences) vesicles (0.7 ng/mL) were prepared according to the method of Hiratsuka et al. (Arch. Oral. Biol. 1982, 37: 717-724), followed by 30 minutes of reaction with each antibody at 37° C. Furthermore, Actinomyces viscosus (A. viscosus, ATCC19246) was anaerobically cultured in 37 mg/mL brain heart infusion (produced by BBL) supplemented with 5 mg/mL yeast extract (produced by BBL) at 37° C. A. viscosus was prepared to have an absorbance of 1.5 (wavelength of 500 nm) using PBS. The thus prepared 50 μL of A. viscosus was suspended in an equivalent amount of PBS. 50 μL of P. gingivalis 381 vesicles that had reacted with each antibody were added to the solution, followed by 10 minutes of reaction on a flocculation slide at 37° C. After reaction, aggregation activity was evaluated via the naked eye or a light microscope. Inhibition activity criteria (score: 0 to 4) were in accordance with the method of Cisar. J. O. et al., (Infect. Immun., 1979 Vol 33: 467). As a result, 13 anti-r40-kDa OMP antibodies showed activity of inhibiting the coaggregation of P. gingivalis 381 vesicles with A. viscosus. From among antibodies having such activity, h13-17, 5-89-2, a44-1, and 1-85-16 having strong coaggregation-inhibiting activity were selected (Table 1). The following experiment was conducted using these 4 antibodies.

TABLE 1
Each anti-40 kDa-OMP antibody's activity of
inhibiting the coaggregation of P. gingivalis
Table 1

| | Activity of inhibiting | |
|---|---|---|
| Clone | coaggregation | Subclass |
| a44-1 | 1 | IgG1(κ) |
| h13-17 | 1 | IgG1(κ) |
| 1-85-16 | 1 | IgG1(κ) |
| 5-89-2 | 2 | IgG1(κ) |

Antibody concentration: a44-1 (193 μg/mL), h13-17 (175 μg/mL), 1-86-16 (161 μg/mL), 5-89-2 (32 μg/mL)
The lower the numerical value representing ability of inhibiting coaggregation, the stronger the activity

### EXAMPLE 10

### Activation of Promoting Human Neutrophilic Phagocytosis by Anti-r40-kDa OMP Antibody

Phagocytosis tests were implemented by a modified version of the method of Perticarari S. et al., (Cytometry 1991; 12: 687-693). The P. gingivalis 381 strain was anaerobically cultured in Tripticase soy broth (produced by BBL) supplemented with 5 μg/mL hemin, 0.5 μg/mL vitamin K, and 0.5% yeast extract (produced by Difco Laboratories) at 37° C. P. gingivalis was grown to the mid-log phase. Subsequently, the bacterial cells were collected by centrifugation (10,000×g, 10 minutes, 4° C.), followed by heat treatment at 60° C. for 30 minutes. After washing twice with PBS, the cells were adjusted to a concentration of 2×10⁸ cfu/mL. 1 mL of FITC (produced by Molecular Probes Inc.,) prepared to a concentration of 1 mg/mL with a 0.1 M sodium carbonate buffer (pH9.6) was added to 1 mL of a solution containing a suspension of P. gingivalis 381, followed by 30 minutes of culture at 37° C. After washing three times with PBS, FITC-labeled P. gingivalis 381 was prepared to a concentration of 2×10⁸ cfu/mL. Binding of the anti-r40-kDa OMP antibody to the FITC-labeled P. gingivalis 381 was at the same level as that for unlabeled P. gingivalis 381. A method for separating human neutrophils was carried out by collecting human peripheral venous blood using a heparin-coated vacuum tube for blood collection and then carrying out double-density gradient centrifugation using Histopaque 1077 and 1119 (produced by Sigma). Furthermore, the remaining erythrocytes were hypotonically hemolysed in ice-cooled red blood cell lysis solution (10 mM Tris, 10 mM KCl, 1 mM MgCl₂, and pH7.4). After recovery of osmotic pressure with PBS, the resultant was washed and then prepared to a concentration of 2×10⁶ /mL of human neutrophils. The thus prepared neutrophils were immediately subjected to the next phagocytosis test.

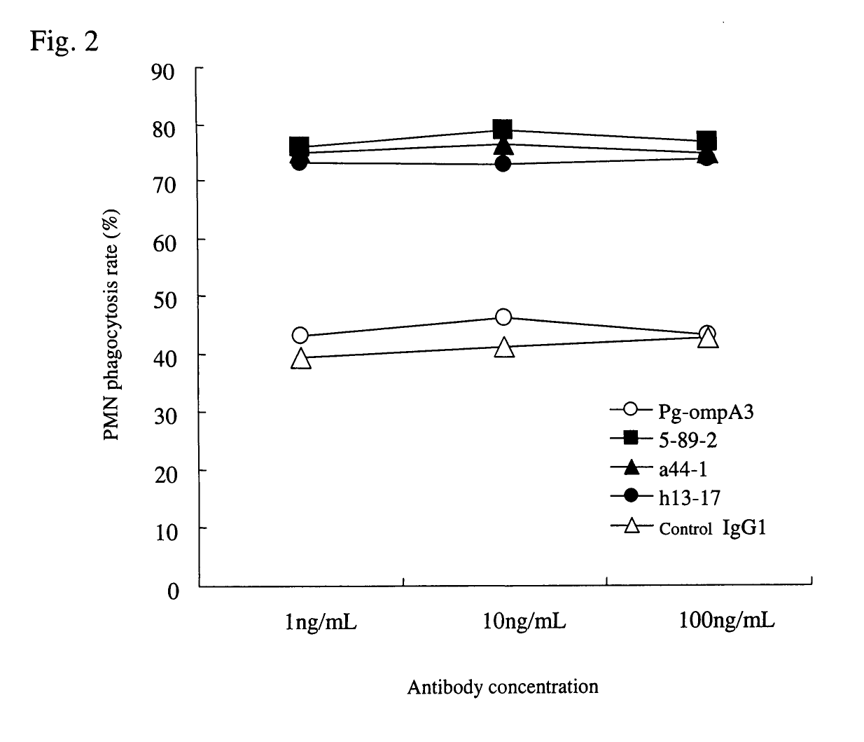

The anti-r40-kDa OMP antibody or a control antibody (1 μg/mL and 5 μL) was added to 2×10⁷ cfu/mL FITC-labeled P. gin givalis 381, followed by 30 minutes of reaction at 37° C. After washing twice with PBS, the resultant was re-suspended. Human neutrophils and FITC-labeled P. gingivalis were mixed at a ratio of 1:20 and then cultured for 30 minutes at 4° C. or 37° C. After phagocytic reaction, 10,000 neutrophils were incorporated by FACScan (produced by Becton, Dickinson and Company) and then the fluorescence intensity of FITC was measured, so that phagocytic ability was evaluated. The percentage of neutrophils (phagocytosis rate) that had conducted phagocytosis was found by the following equation. Phagocytosis rate (%)=(percentage (%) of FITC-positive neutrophils at 37° C.)−(percentage (%) of FITC-positive neutrophils at 4° C.). As a result, it was observed (Table 2) that when compared with the control antibody, all antibodies had an effect of enhancing phagocytic activity of neutrophils. Furthermore, the activity of a mouse 40-kDa OMP antibody (Pg-ompA3 as reported in Sito S. et al., Gen. Pharmacol, 1997 Vol 28: 675) was compared with that of human 40-kDa OMP antibodies (h13-17, 5-89-2, and a44-1) using the same evaluation system. As a result, the mouse antibody Pg-ompA3 was found to have very weak activity of enhancing the phagocytic ability of human neutrophils. Thus, the human antibodies were shown to be superior to this mouse antibody (FIG. 2).

TABLE 2
Each anti-40 kDa-OMP antibody's activity of promoting phagocytosis
of P. gingivalis by human polymorphonuclear neutrophils
Table 2

| Clone | PMN phagocytosis rate |
|---|---|
| 5-89-2 | 76.2% * |
| 1-85-16 | 83.0% * |
| a44-1 | 78.6% * |
| h13-17 | 81.3% * |
| Control IgG1 | 49.8% |

* The PMN (Polymorphonuclear neutrophils) phagocytosis rate was significantly higher than that in the case of the control.

### EXAMPLE 11

### Comparison between r40-kDa OMP Antibody and Patient's Serum in Terms of Activity of Binding to P. gingivalis

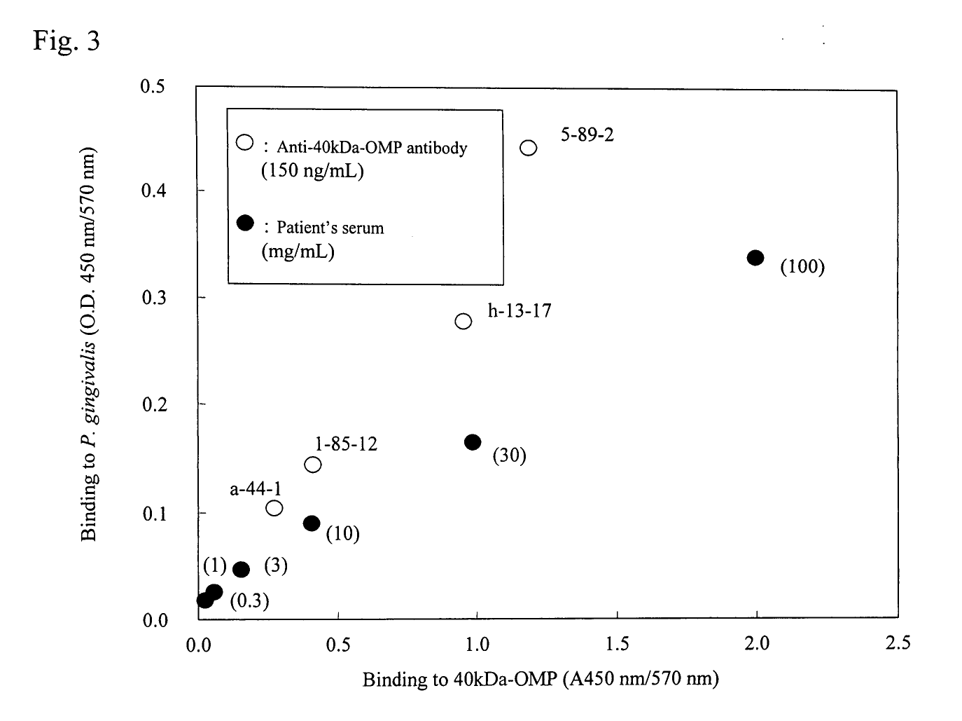

Four r40-kDa OMP antibodies were compared with the sera of chronic periodontal disease patients in terms of the intensity of binding to P. gingivalis by ELISA, the same method employed in Example 4. 50 μl of each antibody (150 μg/mL) or IgG antibody (Kobayashi, T. et al., Infect. Immun., 2001 Vol 69: 2935) derived from the patients' sera that had been appropriately diluted was added to each well of a microplate coated with r40-kDa OMP or ultrasonicated P. gingivalis so that reaction between them could take place. After washing, bound antibodies were detected using peroxidase-labeled goat anti-human IgG F(ab′)² antibody (produced by Immuno-Biological Laboratories Co., Ltd.) and a coloring substrate solution (TMB). As a result, it was revealed that, when concentrations at which the patients' sera had bound to a degree equivalent to that in the case of r40-kDa OMP were compared with the concentrations of r40-kDa OMPs that had bound to P. gingivalis, all antibodies had more intensive activity of binding to P. gingivalis than that of patients' sera (FIG. 3).

### EXAMPLE 12

### Cross-Reactivity of each Anti-r40-kDa OMP Antibody to Human Blood Cells

The cross-reactivity of each monoclonal antibody to human blood cells was examined by FACS analysis. 10 mL of human peripheral blood containing 1 mL of heparin (produced by Novo A/S) was diluted 2-fold with 10 mL of PBS(−). The solution was superposed on 20 mL of a Ficoll-Paque PLUS solution (produced by Amersham Pharmacia Biotech). After 30 minutes of centrifugation at 1500 r.p.m., a mononuclear leukocyte fraction was collected and then washed twice with PBS(−). The thus prepared cells were suspended at a concentration of 2×10⁶/ml in a PBS Staining Buffer (SB) containing 1% rat serum, 0.1% NaN₃, and 2% FCS. The cell suspension (100 μl/well) was dispensed into a 96-well round-bottomed plate (produced by Becton, Dickinson and Company). The h13-17, 5-89-2, a44-1, or 1-85-16 antibody was incubated at a concentration of 5 μg/mL at ice temperature for 30 minutes. After washing twice with SB, the resultant was suspended in 300 μl of a FACS buffer. The reactivity of each antibody was examined by FACS (FACScan produced by Becton, Dickinson and Company). As a result, no antibodies were found to bind to human peripheral blood cells. Based on these results, it was inferred that all antibodies merely cause side effects upon administration to humans.

### EXAMPLE 13

### Binding Activity of Anti-r40-kDa OMP Antibody to P. gingivalis

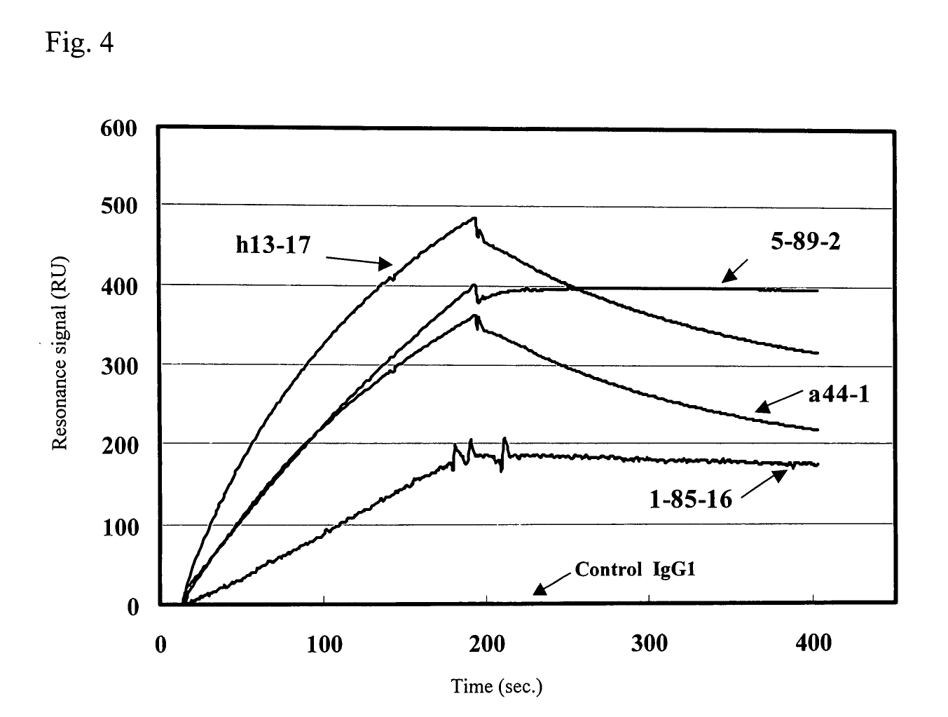

Binding of each antibody to P. gingivalis was evaluated using a surface plasmion resonance method (BIACORE, produced by BIACORE International AB). Sensor chips (CM5) were subjected to immobilization according to experimental protocols by an amine coupling method (Immobilized amounts: cIgG1=9097RU, a44-1=7355RU, h13-17=7473RU, 5-89-2=7595RU, and 1-85-16=7870RU). P. gingivalis was treated at 60° C. for 30 minutes so as to die and then the bacterial bodies were prepared to a concentration of O.D. 550 nm=0.4 with a 10 mM Tris-HCl/150 mM NaCl buffer (pH 8.0). The resultants were supplied at a flow rate of 10 μL/min. As a result, the dissociation rate in the case of 5-89-2 was slower than those of the other antibodies, showing that the 5-89-2 antibody strongly binds to P. gingivalis (FIG. 4). In view of saliva's action to eliminate antibodies that will be exerted upon treatment of periodontal diseases, a preferable antibody is the 5-89-2 antibody showing slow dissociation, for example. Therefore, it was inferred that the 5-89-2 antibody shows a highly therapeutic effect when it is administered to a human.

### EXAMPLE 14

### Anti-r40-kDa OMP Antibodies' Activity of Inhibiting the Binding of Hemin to P. gingivalis

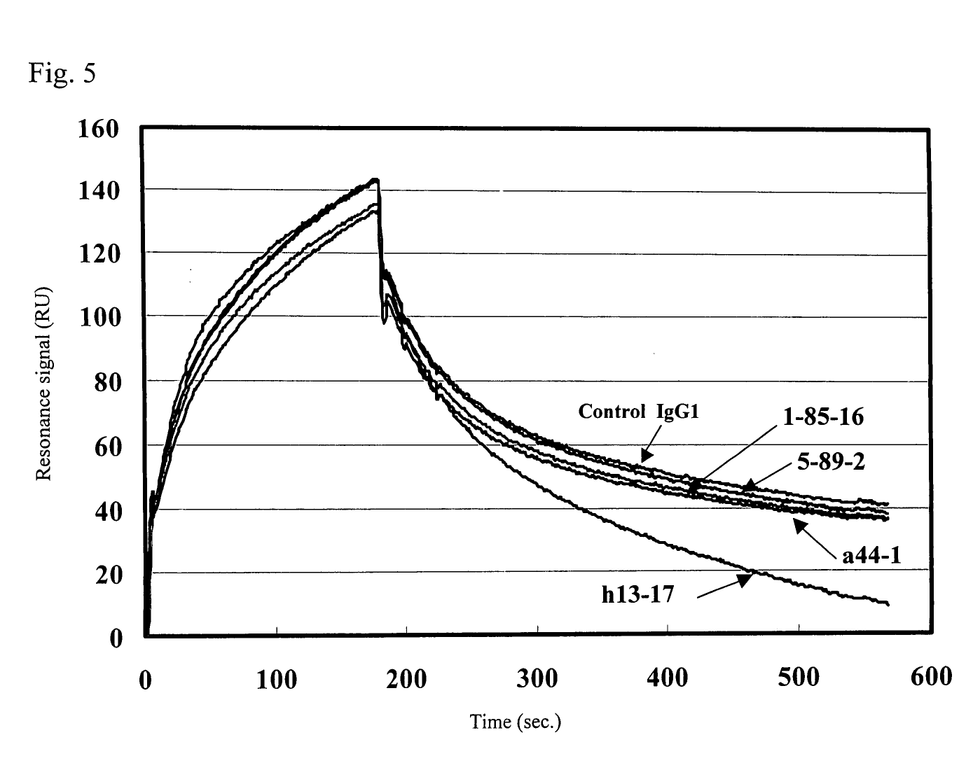

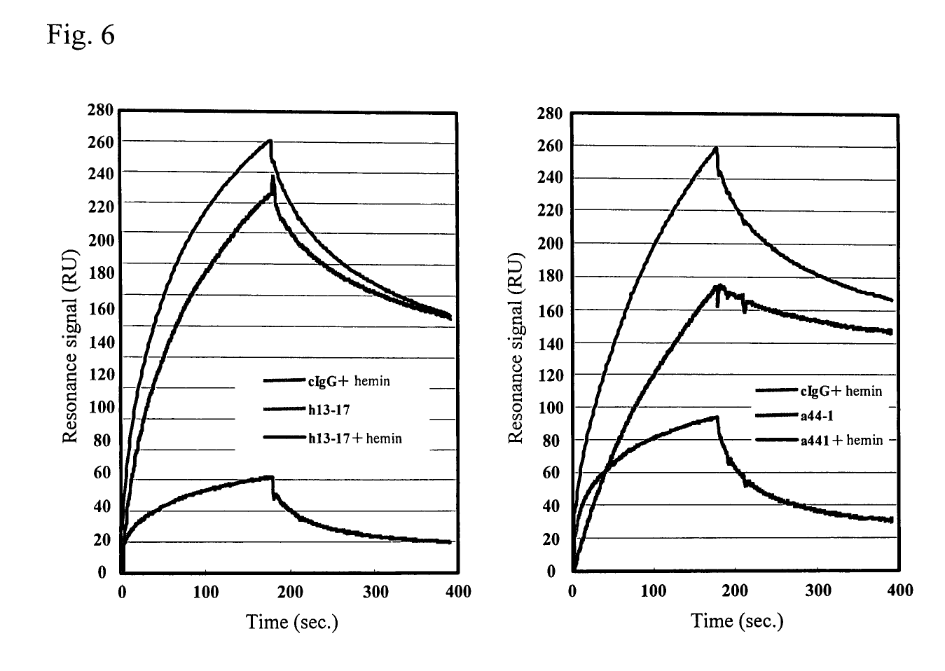

Whether or not anti-r40-kDa OMP antibodies inhibit the binding of hemin to 40-kDa OMP was evaluated using a surface plasmon resonance method (BIACORE, produced by BIACORE International AB). r40-kDa OMP was immobilized by an amine coupling method (RU=3500). FIG. 5 shows the results of supplying each antibody prepared at 20 μg/mL with a 10 mM Tris-HCl/150 mM NaCl (T-B, pH 8.0) buffer at a flow rate of 20 μL/m and then supplying 5 μg/mL hemin (produced by Sigma) prepared with a T-B buffer. FIG. 5 shows a sensorgram where the time when the supply of hemin was initiated was determined to be 0 seconds. When h13-17 had been previously bound to r40-kDa OMP, significantly less binding of hemin was observed compared with the case of cIgG. On the other hand, in the case of the other 3 clones, binding of r40-kDa OMP to hemin was observed to the same degree as that in the case of a control, even when they had been previously bound to r40-kDa OMP. These results suggested that h13-17 inhibits the binding of hemin to r40-kDa OMP. Furthermore, FIG. 6 shows the results of examining whether or not h13-17 inhibits the binding of hemin to r40-kDa OMP when h13-17 and hemin are simultaneously caused to react. h13-17 and hemin were prepared to concentrations of 20 μg/mL and 5 μg/mL, respectively, with a T-B buffer. They were supplied to r40-kDa OMP-immobilized sensor chips at a flow rate of 20 μL/m. a44-1 antibody having a dissociation rate that is almost the same (see FIG. 4) as that of h13-17 was used as a control antibody. As a result, when signals at a time (380 seconds) near the time of the equilibrium after diffusion of analytes were observed, the binding signal of hemin to r40-kDa OMP was 255 RU in the presence of h13-17, which was almost the same as that (252 RU) in the case of h13-17 alone. The difference in the signals (255 RU−252 RU=3 RU) was clearly lower than the signal (30 RU) in the case of control IgG 1+hemin at this time, suggesting a lack of binding of hemin to r40-kDa OMP. On the other hand, regarding the signal in the case of the control a44-1 at 380 sec., the sum of the signal in the case of a44-1 and the signal in the case of control IgG1+hemin was 180 RU (148 RU+32 RU). The signal in the case of a44-1+hemin was 170 RU, which was close to the theoretical value (180 RU) at the time when no antibodies were competing with hemin. These results suggested that the h13-17 antibody strongly inhibits the binding of hemin to OMP40.

### EXAMPLE 15

### Suppressive Effect of Anti-r40-kDa OMP Antibodies on Rat Experimental Periodontitis

Rat neutrophils were separated from rat peripheral blood by density gradient centrifugation using a Lympholite-Rat (produced by Sigma). In a manner similar to Example 10, the activity of promoting rat neutroplilic phagocytosis of each anti-r40-kDa OMP antibody was evaluated. Thus the activity was observed. Hence, an experimental system for periodontitis was prepared by inoculating rat oral cavities with P. gingivalis to induce alveolar bone resorption. Through the use of such system, the effect of the anti-r40-kDa OMP antibodies (having strong ability to bind to, activity of inhibiting coaggregation ability of, and activity of promoting neutrophilic phagocytosis of P. gingivalis) to suppress rat experimental periodontitis was examined in in vitro experiments. 3-week-old Sprague-Dawley SPF rats (6 rats per group) were used for induction and infection of rat experimental periodontitis due to inoculation with P. gingivalis. After observation of health conditions, sulfamethoxazole with a final concentration of 1 mg/ml was mixed with 200 μg/ml trimethoprim in ion exchanged water. The mixed solution was administered as drinking water for 1 week, thereby reducing normal bacteria of the mouth. Subsequently, antibiotic-free ion exchanged water was administered for 3 days. A bactereial solution (10⁹CFU/ml) of P. gingivalis strain (ATCC33277) was prepared with a 5% carboxy methyl cellulose (CMC) solution prepared with PBS. The bacterial solution was directly administered into rat oral cavities 3 times every 2 days. A group (sham) that had not been inoculated with P. gingivalis was administered with only 5% CMC solution and raised under the same condtions. Antibodies were administered as follows. a44-1, h13-17, and 5-89-2 antibodies were each prepared to a concentration of 0.5 mg/ml. 3 antibodies were mixed in equal amounts, the resultant was diluted with a 5% CMC solution, and then the solution was administered to a group. A DNP human antibody (IgG1) prepared to be a concentration of 0.5 mg/ml with a 5% CMC solution prepared using PBS was administered to a control group. These groups were used. Administration took place 9 times in total; specifically, administration was performed every day from 2 days before the start of administration of P. gingivalis to 2 days after the final administration. In addition, at the time of inoculation using P. gingivalis, the antibodies were administered 10 minutes after inoculation with the bacteria. All rats were fed ad libitunm diets and drinking water and raised under conditions of 12-hour light and dark cycles at a temperature of 23° C. and humidity of 60%.

The presence of P. gingivalis in rat oral cavivies was confirmed by performing PCR reaction. Specifically, each rat oral cavity was wiped with a swab for 30 seconds to collect plaque bacteria. DNA was extracted using ISOPLANT (produced by NIPPON GENE CO., LTD.). The extracted DNA was dissolved in 20 μL of a T₁₀E₁ solution and then stored. Primers were prepared based on the nucleotide sequence of 16S rRNA according to the report of Ashimoto et al., (Ashimoto, A. et al., Oral Microbiol Immunol., 1996 Vol 11: 266). PCR reaction was performed, after heating at 95° C. for 5 minutes for denaturation, in 35 cycles, each of which conisted of 95° C. for 30 seconds, 60° C. for 1 minute, and 72° C. for 1 minute.

Bone resorption was measured as follows. On day 42 after the final inoculatoin with P. gingivalis, all rats were sacrified by decapitation and bloodletting under ether anesthesia. The amounts of alveolar bone resorption were evaluated by measuring distances (seven positions) between the cement-enamel junction of a maxillary molar tooth portion and the crest of the alveolar ridge. The cranial bones were heated under an atmospheric pressure of 2 for 10 minutes and immersed in a 3% sodium hypochlorite solution to remove soft tissues. The alveolar bones were stained and dried with a 1% methylene blue solution. The thus prepared samples were measured under a digital HD microscope (stereoscopic microscope produced by KEYENCE CORPORATION) at ×40 magnification. Values measured at 7 positions were averaged to obtain the bone resorption amount per rat individual. The mean value obtained by averaging the mean values of 6 rats was determined to represent the bone resorption amount of an experimental group and denoted with milimeters. Measurement was carried out three times for the same sample and then the mean value and standard error (SE) were found. Statistic analysis was carried out using Fisher's PLSD (StatView).

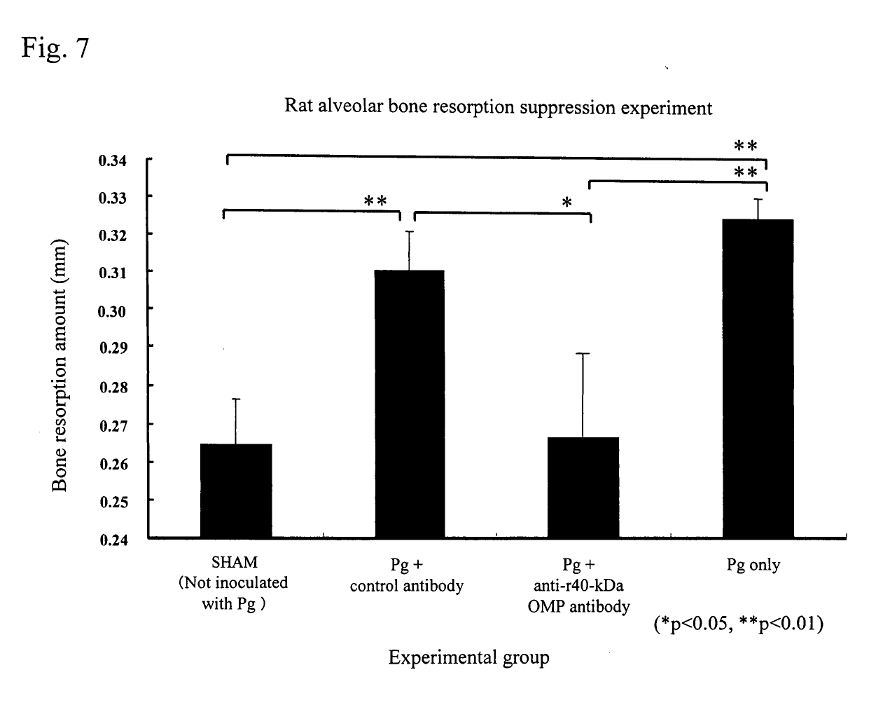

As a result (FIG. 7), a clearly significant increase was observed in bone resorption amount (p<0.01) in the case of a group to which P. gingivalis had been administered, compared with a group to which none had been administered. Furthermore, significantly suppressed bone resorption was confirmed by the administration of the anti-r40-kDa OMP antibodies. Specifically, the amount of bone resorption in the case of a group for which the anti-r40-kDa OMP antibodies had been used at a concentration of 0.5 mg/ml was almost the same as that in the case of a group to which no P. gingivalis had been administered and in which almost no bone resorption due to administration of P. gingivalis was observed. In the case of a group to which the control antibody (DNP antibody) had been administered, the amount of bone resorption was somewhat decreased compared with a group to which only P. gingivalis had been administered. However, no significant difference was observed between the two. Based on the above results, it was revealed that in this experiment using bone resorpton as an index, monoclonal antibodies against the OMP40 antigen can suppress rat alveolar bone resorption. Furthermore, clear decreases were observed in the amounts of bone resorption with the administration of the OMP40 antibodies. Accordingly, the remaining rates of P. gingivalis in the rat oral cavities at the completion of this experiment were examined by PCR. As a result, in the case of the group that had been inoculated with P. gingivalis, the presence of P. gingivalis was confirmed in all six rats. However, in the case of the group to which the OMP40 antibody had been administered, the presence of P. gingivalis was not confirmed in 5 out of 6 rats (Table 3).

TABLE 3
Detection of P. gingivalis from rat oral cavities by PCR
Table 3 Detection of P. gingivalis from rat oral cavities by PCR

| Experimental group | Detection rate (%) |
|---|---|
| Not inoculated with P.g, Pg+ | 0/6 (0) |
| Pg + control antibody | 5/6 (83.3) |
| Pg + anti-r40-kDa OMP antibody | 1/6 (16.7) |
| Pg only | 6/6 (100) |

### INDUSTRIAL APPLICABILITY

As described in Examples, the antibody binding to 40-kDa OMP of the present invention inhibits the aggregation of P. gingivalis and promotes the phagocytic ability of leukocytes. Furthermore, the antibody binding to 40-kDa OMP of the present invention can eliminate P. gingivalis from the oral cavity. Accordingly, the antibody or the functional fragment thereof of the present invention can be effectively used for treating and diagnosing periodontal diseases.

All publications cited herein are incorporated herein in their entirety. A person skilled in the art would easily understand that various modifications and changes of the present invention are feasible within the technical idea and the scope of the invention as disclosed in the attached claims. The present invention is intended to include such modifications and changes.

1. An antibody binding to 40-kDa OMP or a functional fragment thereof, which has activity of inhibiting the binding of hemin to 40-kDa OMP.

2. An antibody binding to 40-kDa OMP or a functional fragment thereof, which has (1) activity of inhibiting the coaggregation of P. gingivalis and (2) activity of promoting human neutrophilic phagocytosis.

3. An antibody binding to 40-kDa OMP or a functional fragment thereof, which has (1) activity of inhibiting the coaggregation of P. gingivalis and (2) activity of inhibiting the binding of hemin to 40-kDa OMP.

4. An antibody binding to 40-kDa OMP or a functional fragment thereof, which has (1) activity of promoting human neutrophilic phagocytosis and (2) activity of inhibiting the binding of hemin to 40-kDa OMP.

5. An antibody binding to 40-kDa OMP or a functional fragment thereof, which has (1) activity of inhibiting the coaggregation of P. gingivalis, (2) activity of promoting human neutrophilic phagocytosis, and (3) activity of inhibiting the binding of hemin to 40-kDa OMP.

6. The antibody or the functional fragment thereof according to any one of claims 2, 3, and 5, wherein the coaggregation of P. gingivalis is coaggregation of P. gingivalis and Actinomyces viscosus.

7. An antibody binding to 40-KDa OMP or a functional fragment thereof, which has activity of suppressing alveolar bone resorption.

8. The antibody or the functional fragment thereof according to any one of claims 1 to 7, wherein the antibody is a human antibody.

9. The antibody or the functional fragment thereof according to any one of claims 1 to 8, which is produced by a mouse-mouse hybridoma.

10. The antibody or the functional fragment thereof according to any one of claims 1 to 9, wherein the antibody is a monoclonal antibody.

11. The antibody or the functional fragment thereof according to any one of claims 1 to 10, which covalently or non-covalently binds to a therapeutic agent.

12. The antibody or the functional fragment thereof according to claim 11, wherein the therapeutic agent is selected from antibiotics or antibacterial agents.

13. The antibody or the functional fragment thereof according to claim 12, wherein the antibiotic or the antibacterial agent is tetracycline or minocycline.

14. The antibody or the functional fragment thereof according to any one of claims 1 to 13, wherein the antibody class is IgG.

15. The antibody or the functional fragment thereof according to claim 14, wherein IgG is IgG1.

16. The antibody or the functional fragment thereof according to any one of claims 1 to 13, wherein the antibody class is IgA.

17. The antibody or the functional fragment thereof according to any one of claims 1 to 16, wherein the amino acid sequence of a heavy chain constant region is altered.

18. An antibody binding to 40-kDa OMP or a functional fragment thereof, which is produced by a hybridoma h13-17 (accession No. FERM BP-8325).

19. An antibody binding to 40-kDa OMP or a functional fragment thereof, which comprises variable regions of an antibody that is produced by a hybridoma h13-17 (accession No. FERM BP-8325).

20. The antibody or the functional fragment thereof according to claim 18 or 19, which covalently or non-covalently binds to a therapeutic agent.

21. The antibody or the functional fragment thereof according to claim 20, wherein the therapeutic agent is selected from antibiotics or antibacterial agents.

22. The antibody or the functional fragment thereof according to claim 21, wherein the antibiotic or the antibacterial agent is tetracycline or minocycline.

23. The antibody or the functional fragment thereof according to any one of claims 18 to 22, wherein the antibody class is IgG.

24. The antibody or the functional fragment thereof according to claim 23, wherein IgG is IgG1.

25. The antibody or the functional fragment thereof according to any one of claims 18 to 22, wherein the antibody class is IgA.

26. The antibody or the functional fragment thereof according to any one of claims 18 to 25, wherein the amino acid sequence of a heavy chain constant region is altered.

27. A hybridoma h13-17 (accession No. FERM BP-8325).

28. An antibody binding to 40-kDa OMP or a functional fragment thereof, which is produced by a hybridoma 5-89-2 (accession No. FERM BP-8323).

29. An antibody binding to 40-kDa OMP or a functional fragment thereof, which comprises variable regions of an antibody that is produced by a hybridoma 5-89-2 (accession No. FERM BP-8323).

30. The antibody or the functional fragment thereof according to claim 28 or 29, which covalently or non-covalently binds to a therapeutic agent.

31. The antibody or the functional fragment thereof according to claim 30, wherein the therapeutic agent is selected from antibiotics or antibacterial agents.

32. The antibody or the functional fragment thereof according to claim 31, wherein the antibiotic or the antibacterial agent is tetracycline or minocycline.

33. The antibody or the functional fragment thereof according to any one of claims 28 to 32, wherein the antibody class is IgG.

34. The antibody or the functional fragment thereof according to claim 33 wherein IgG is IgG1.

35. The antibody or the functional fragment thereof according to any one of claims 28 to 32, wherein the antibody class is IgA.

36. The antibody or the functional fragment thereof according to any one of claims 28 to 35, wherein the amino acid sequence of a heavy chain constant region is altered.

37. A hybridoma 5-89-2 (accession No. FERM BP-8323).

38. An antibody binding to 40-kDa OMP or a functional fragment thereof, which is produced by a hybridoma a44-1 (accession No. FERM BP-8324).

39. An antibody binding to 40-kDa OMP or a functional fragment thereof, which comprises variable regions of an antibody that is produced by a hybridoma a44-1 (accession No. FERM BP-8324);

40. The antibody or the functional fragment thereof according to claim 40 or 41, which covalently or non-covalently binds to a therapeutic agent.

41. The antibody or the functional fragment thereof according to claim 40, wherein the therapeutic agent is antibiotics or antibacterial agents.

42. The antibody or the functional fragment thereof according to claim 41, wherein the antibiotic or the antibacterial agent is tetracycline or minocycline.

43. The antibody or the functional fragment thereof according to any one of claims 38 to 42, wherein the antibody class is IgG.

44. The antibody or the functional fragment thereof according to claim 43, wherein IgG is IgG1.

45. The antibody or the functional fragment thereof according to any one of claims 38 to 42, wherein the antibody class is IgA.

46. The antibody or the functional fragment thereof according to any one of claims 38 to 45, wherein the amino acid sequence of a heavy chain constant region is altered.

47. A hybridoma a44-1 (accession No. FERM BP-8324).

48. A nucleic acid, which is possessed by a hybridoma selected from the group consisting of a hybridoma h13-17 (accession No. FERM BP-8325), a hybridoma 5-89-2 (accession No. FERM BP-8323), and a hybridoma a44-1 (accession No. FERM BP-8324) and encodes an antibody containing a variable region of an antibody produced by the hybridoma or a functional fragment of the said antibody.

49. A protein encoded by the nucleic acid according to claim 48, which is an antibody or a functional fragment thereof.

50. An expression vector, which has the nucleic acid according to claim 48.

51. A host, which has the expression vector according to claim 50.

52. The host according to claim 51, which is selected from the group consisting of Escherichia coli, yeast cells, insect cells, mammalian cells, plant cells, and mammals.

53. A method for producing an antibody binding to 40-kDa OMP, which comprises isolating a gene that encodes an antibody binding to 40-kDa OMP from a hybridoma selected from the group consisting of a hybridoma h13-17 (accession No. FERM BP-8325), a hybridoma 5-89-2 (accession No. FERM BP-8323), and a hybridoma a44-1 (accession No. FERM BP-8324), constructing an expression vector comprising the gene, introducing the expression vector into a host to cause expression of the antibody, and collecting the antibody from the obtained host, the culture supernatant of the host, or secretion from the host.

54. An agent for suppressing alveolar bone resorption, which contains an antibody binding to 40-KDa OMP or a functional fragment thereof as an active ingredient.

55. An agent for preventing, diagnosing, or treating periodontal diseases, which contains an antibody binding to 40-kDa OMP or a functional fragment thereof as an active ingredient.

56. Use of an antibody binding to 40-KDa OMP or a functional fragment thereof for production of an agent for suppressing alveolar bone resorption.

57. A method for suppressing alveolar bone resorption, which comprises preparing an antibody binding to 40-KDa OMP or a functional fragment thereof and administering the antibody or the fragment to an animal.

58. Use of an antibody binding to 40-KDa OMP or a functional fragment thereof for production of an agent for preventing, diagnosing, or treating periodontal diseases.

59. A method for diagnosing, preventing, or treating periodontal diseases, which comprises preparing an antibody binding to 40-KDa OMP or a functional fragment thereof and administering the antibody or the fragment to an animal.

60. An agent for preventing, diagnosing, or treating periodontal diseases, which contains the antibody or the functional fragment thereof according to any one of claims 1 to 26, 28 to 36, 38 to 46, and 49 as an active ingredient.

61. An agent for suppressing alveolar bone resorption, which contains the antibody or the functional fragment thereof according to any one of claims 1 to 26, 28 to 36, 38 to 46, and 49 as an active ingredient.

62. Use of the antibody or the functional fragment thereof according to any one of claims 1 to 26, 28 to 36, 38 to 46, and 49 for production of an agent for preventing, diagnosing, or treating periodontal diseases;

63. Use of the antibody or the functional fragment thereof according to any one of claims 1 to 26, 28 to 36, 38 to 46, and 49 for production of an agent for suppressing alveolar bone resorption.

64. A method for diagnosing, preventing, or treating periodontal diseases, which comprises preparing the antibody or the functional fragment thereof according to any one of claims 1 to 26, 28 to 36, 38 to 46, and 49 and administering the antibody or the fragment to an animal.

65. A method for suppressing alveolar bone resorption, which comprises preparing the antibody or the functional fragment thereof according to any one of claims 1 to 26, 28 to 36, 38 to 46, and 49 and administering the antibody or the fragment to an animal.


SEQUENCE LISTING (2 sequences)

SEQ ID NO: 1 - DNA, 2008 bp, Porphyromonas gingivalis
gaattccaaccgtggaaaggggagatagagcggcgcaacaacggatcgatcattgcgctcgaaagcggaacggcctatgcctatgccctgaataatctccaaagccgtggacgcttcttcatctctccgcaggaggaggtctatgccggtcaggtcgtgggcgagcacacgaaggagggcgatctctgcgtcaacgtctgcaagagcaaaaagcttaccaatatgcgtgcttccggtagcgatgataaggtgtcactggctccaccggtggtattcagcctcgaagatgctttggagtatatcaagtatgacgagtatgtggaggtgacgccgaaaagcatgcgcatgcgtaaaattatcctcgacgagacagaacgtaaacgacaaggacgataacagaggcaaagtatttgcagatttcatctatctaacgtattttggtgcccgaaatacggaaaatcaaacaaaatgtttcactaacaaaaaagataaacgaacatgaaaagattattactctctgctgctatcctaagtagtatggctttgtttaatgtcaatgcacaagagttgaaaacctctgctgacatgaaaggttcttttaagaagaatgtggtattggaggtatttactgccgaatggtgcggttactgtccaggtggtaaagagcgcattgcaaaagcaattgaaatgttggatgatgaatataaggagcgtgtttttcagacatttgttcattataatgatgggatctcaaaaaaatggcctcgtgttggccaacttttcattgcattggatcaaacattgggcattccgggttttccgactttttcagtttgccgtatggagaaaaaaggtgaaaatctttcaataggtgctccaatagcaattaaaaataagattatgaaaggttttggtgatggtacagcccctgcagaggtaaaccttaaattgaccaaaggtgcaacaccggaagatgtatgtacagctacatttactggtaaagtcgatgcagacctcatagggaaacctcttatgttgactgcatatgtattgaaaaacaatatgaagcctattaatccgcaaaatggagctggggatggatatctccaccaacatactgtgttaatgattctctccacagatgtaaaaggagacgctttaaatattgcagccgatggaagttttaccatcaagaaagaatttaagttggatggctttgaaataaaagatacagatgttcttgctttcgtacaccatccaatgtccaatgcggaaaaccattctattatcaatgccgggcaagaaagccttgataaagcagagcctacagctacagaacaaattgttgctaccccctctgtcaaagcatatgttcagaatggcaaaattgttgtagaggaagagtattccaagatggaagtattcaatgcaactggtcaacttgtcaaaaatgaatcccttgtccccggtgtctatgttgtccgtataacggcaaacggtgtaatgcatttccttaaagtcttagttccttgatttattgagctaagatttaaacgaaaactgcgccttcttaatgtttcataagaaggcgcagtttccttttttcctcattccattctccgggtggtcgtcgaaggggactgcctgtcatctttcaaacgaggaatatcagctcccctcacccaacacgatgtccacaaatccatgttcatagcctatttgagacctttgaaaaaagagaaggtttcttatatctattatctataaaggcactaaaaatcagtcgattgacaataaaatctccaagaaaataccatgcgacagactcactatatagatgcggtcaatggtctgatatatcgaggctgttgtggatggaggatagtacacatagttttgcacctgaaacaaacgttcatactaaactaaaaacaaaagcattatgactcctatcctgaacaccgttttccccgagttcaaactcaatgcctatcacaatggcgaattc

SEQ ID NO: 2 - protein, 344 aa, Porphyromonas gingivalis
MKRLLLSAAILSSMALFNVNAQELKTSADMKGSFKKNVVLEVFTAEWCGYCPGGKERIAKAIEMLDDEYKERVFQTFVHYNDGISKKWPRVGQLFIALDQTLGIPGFPTFSVCRMEKKGENLSIGAPIAIKNKIMKGFGDGTAPAEVNLKLTKGATPEDVCTATFTGKVDADLIGKPLMLTAYVLKNNMKPINPQNGAGDGYLHQHTVLMILSTDVKGDALNIAADGSFTIKKEFKLDGFEIKDTDVLAFVHHPMSNAENHSIINAGQESLDKAEPTATEQIVATPSVKAYVQNGKIVVEEEYSKMEVFNATGQLVKNESLVPGVYVVRITANGVMHFLKVLVP